(19) United States
(12) Patent Application Publication
(10) Pub. No.: US 2014/0248374 A1
(43) Pub. Date: Sep. 4, 2014

(54) COMPOSITIONS AND MULTI-STEP METHODS OF USING THE SAME FOR THE TREATMENT OF JELLYFISH STINGS

(71) Applicant: Angel Anne YANAGIHARA, Honolulu, HI (US)

(72) Inventor: Angel Anne YANAGIHARA, Honolulu, HI (US)

(73) Assignee: UNIVERSITY OF HAWAII, Honolulu, HI (US)

(21) Appl. No.: 13/401,640

(22) Filed: Feb. 21, 2012

Related U.S. Application Data
(60) Provisional application No. 61/444,656, filed on Feb. 18, 2011.

(51) Int. Cl. A61K 33/34; A61K 31/17; A61K 33/06; A61K 33/30; A61K 31/7016

(52) U.S. Cl. CPC A61K 33/34; A61K 33/30; A61K 31/7016; A61K 33/06; A61K 31/17
USPC 424/638; 424/630

(57) ABSTRACT

Embodiments of the invention relate to a composition containing zinc and copper and methods of using the same in treatment of exposure to a pore-forming toxin (PFT) in a subject or membrane perturbant (MP).

### CROSS-REFERENCE TO RELATED APPLICATIONS

This application claims priority from U.S. Provisional Application Ser. No. 61/444,656, filed on Feb. 18, 2011, which is incorporated herein by reference in its entirety.

### STATEMENT REGARDING FEDERALLY-SPONSORED RESEARCH

This invention was supported in part with government support under Grant No. U54 NS039406, awarded by the National Institutes of Health; under Grant No. G12 RR003061, awarded by the National Institutes of Health; under Grant No. R21 19DA024444, awarded by the National Institutes of Health; and under Grant No. P20 RR016453, awarded by the National Institutes of Health. The government has certain rights in the invention.

### FIELD OF THE INVENTION

The invention disclosed herein generally relates to compositions and multi-step methods of using the same to treat conditions caused by exposure to a pore-forming toxin.

### BACKGROUND

Each year hundreds of thousands of beachgoers are stung by cnidarian family members, which include the anthozoan (anemone, corals), hydrozoans, scyphozoans (common jellyfish), and cubozoans (tropical cuboidal jellies with venoms that result in high morbidity and mortality). For example, life-threatening Chironex fleckeri envenomations occur each year, from November to May, in North Queensland, Australia. There is no clearly effective specific therapy available. Current treatments are directed at relief of symptoms or, in serious cases, support of cardiovascular integrity after envenomation. A few currently available “Sting Relief” type sprays are typically comprised of ingredients such as vinegar, lidocaine, papain, aloe, eucalyptus oil, and menthol. Thus, there is a need for effective therapies to reduce the morbidity and mortality outcomes for cnidarian envenomations, which include addressing inflammation, burning pain, and systemic and cardiovascular outcomes related to cnidarian envenomations.

In addition, pore-forming toxins, or porins, represent an ancient and conserved toxic exudate of most pathogenic bacteria, including staphylococci, streptococci, anthrax, Clostridium, and E. coli, and are a major constituent of many marine and insect venoms, including the venoms of bees and certain spiders. Potent membrane disruptive porins allow the evasion of host phagocytosis in bacterial infection and rapid prey cytolysis in invertebrate envenomation. They constitute a fundamental mechanism for infection and prey capture. Thus, effective therapies for treatment of cnidarian envenomations will have general applicability to all conditions associated with pore-forming toxins (PFTs) as well as more broadly to pathologies resulting from other membrane perturbants (MPs).

### SUMMARY OF THE INVENTION

Embodiments of the invention are directed to compositions for use in methods for treating a subject suffering from a disease, illness, syndrome or condition resulting from the action of PFTs or other MPs.

Embodiments of the invention are directed to compositions including zinc and copper in a form and an amount effective for the treatment of membrane perturbation. In some embodiments, the zinc can include, for example, a zinc-containing compound that includes a non-toxic counter-ion to zinc. In some embodiments, the copper can include, for example, a copper-containing compound that includes a non-toxic counter-ion to copper. In some embodiments, the counter-ion can include, for example, but not limited to, acetate, malate, D-lactulose, glucose, lactose, galactose, sucrose, pentose, fructose, chloride, sulfate, phosphate, acetate, propionate, butyrate, oxalate, malonate, succinate, gluconate, a complex polyanion, and the like.

In some embodiments, the composition further includes at least one of lactulose, magnesium, and urea. In some embodiments, the lactulose can include, for example, d-lactulose. In some embodiments, the magnesium can include, for example, magnesium sulfate.

In some embodiments, the membrane perturbation includes, for example, exposure to a pore-forming toxin. In some embodiments, the membrane perturbation includes, for example, but not limited to, at least one of: cnidarian envenomation, sea urchin impalement, psoriasis, a dermal inflammatory reaction to an alkaloid of an insect venom or a toxic plant, and the like.

Embodiments of the invention disclosed herein are directed to methods of treating exposure to a pore-forming toxin (PFT) in a subject. The methods include, administering a therapeutically effective amount of any of the compositions disclosed herein, wherein administration of a therapeutically effective amount of the composition results in the treatment of a symptom or a condition resulting from PFT exposure.

Some embodiments of the invention disclosed herein relate to the use of a therapeutically effective amount of any of the compositions disclosed herein in the manufacture of a medicament for the treatment of PFT exposure.

Embodiments of the invention are also directed to a method for treating a mammal suffering from a disease, illness, syndrome or condition resulting from the action of PFTs or other MPs, including administering to the mammal a therapeutically effective dosage of a composition containing a zinc compound and/or a copper compound. In some embodiments, the composition is administered topically. In some embodiments, the composition is administered via transdermal patch.

In some embodiments, the zinc compound is zinc gluconate.

In some embodiments, the copper compound is copper gluconate.

In some embodiments, the disease or condition can be, for example, bacterial sepsis, Irukandji syndrome, cardiovascular collapse, pulseless electrical activity (PEA) hyperkalemia, hemolysis, excessive cytokine and/or histamine release, catecholamine surge, and the like, as well as other dermal inflammatory conditions resulting from MPs.

In some embodiments the disease or condition can be pathologies resulting from membrane perturbant class toxins, such as spider venom small molecular weight compounds or membrane disruptive enzymes such as proteases or lipases, often comprising venoms of invertebrates, as well as fish, snakes, reptiles, amphibians, and mammals.

In some embodiments the disease or condition can be pathologies resulting from skin irritants, including for example, plant alkaloid driven dermal pathologies such as poison ivy, poison oak, or poison sumac.

In some embodiments, the disease or condition can be dermatitis.

In some embodiments, the composition includes d-lactulose.

In some embodiments, the composition includes magnesium sulfate.

In some embodiments, the composition includes urea.

In embodiments of the invention, a method of treating a mammal suffering from a condition resulting from the action of a PFT or MP is provided, wherein the method includes: administering to the mammal a therapeutically effective dose of a composition containing a zinc compound and a copper compound, and applying heat to the afflicted area, wherein administration of the composition and application of heat to the afflicted area results in treatment of the condition. In some embodiments, application of heat is conducted by immersion of the afflicted area in heated water or treatment solution.

Embodiments of the invention are also directed to the use of a zinc compound and a copper compound for the manufacture of a medicament for treating a condition associated with a PFT or MP.

### BRIEF DESCRIPTION OF THE DRAWINGS

FIG. 1 is a graphical representation of the combinatorial inhibition of zinc gluconate and copper gluconate on human RBC hemolysis induced by a cubozoan venom (Alatina moseri, previously described as Carybdea alata) in 2% RBC.

FIG. 2 is a photograph illustrating a comparison of human RBC agar after incubation with a live Alatina moseri tentacle at 37° C. surface temperature in the presence or absence of an exemplary composition as disclosed herein and a commercially available product (“Jelly Squish”).

FIG. 3 is a photograph illustrating a comparison of live Alatina moseri tentacle at 37° C. surface temperature on skin with subsequent sting treatment of an exemplary composition as disclosed herein and a commercially available product (“Jelly Squish”).

FIG. 4 is a graphical representation of point pain intensity as a function of time after skin sting treatment with an exemplary composition as disclosed herein or a commercially available product (“Jelly Squish”).

### DETAILED DESCRIPTION OF THE INVENTION

Pore-forming toxins (PFTs, also known as porins, cytolysins or hemolysins) represent an ancient and conserved toxic constituent of most cnidarian venoms. In addition to inclusion in the venom of cnidarian family members, porins are also a tool of infection used by pathogenic bacteria, including staphylococci, streptococci, anthrax, Clostridium, and E. coli. Potent membrane disruptive porins allow the evasion of host phagocytosis in bacterial infection and rapid prey cytolysis in invertebrate envenomation. They can constitute a fundamental mechanism for infection and prey capture. The venoms of all cubozoans, including two of the most notorious, the lethal Australia box jelly, Chironex fleckeri, and the painful Hawaiian box jelly, Alatina moseri (previously described as Carybdea alata), contain potent porins, lipases and cocktails of bioactive small molecules that drive robust hemolytic and inflammatory responses.

Porin structure and pore formation have been characterized by negative stain electron microscopy and other biochemical techniques that demonstrate the transition of plasma soluble, monomeric forms of these toxins to polymerize to form oligomeric transmembrane pores remarkably comparable to the oligomeric form of human complement C9 (Borsos et al. 1964. “Lesions in erythrocyte membranes caused by immune haemolysis.” Nature 202:251-252; Bhakdi, S, and Tranum-Jensen, J. 1985. “Membrane damage by channel-forming proteins: staphylococcal alpha-toxin, streptolysin-O and the C5b-9 complement complex.” Biochem. Soc. Symp. 50:221-233, each of the foregoing which is incorporated herein by reference in its entirety) as well as the human perforin, the cytolytic protein of cytotoxic T-cells (Young, et al. 1986. “The ninth component of complement and the pore-forming protein (perforin 1) from cytotoxic T cells: structural, immunological, and functional similarities.” Science 233:184-190, which is incorporated herein by reference in its entirety).

Porin insertion compromises the permeability barrier of the cell membrane (Bashford, et al. 1985. “Sequential onset of permeability changes in mouse ascites cells induced by Sendai virus.” Biochim. Biophys. Acta 814:247-255; Bashford, et al. 1986. “Membrane damage by hemolytic viruses, toxins, complement, and other cytotoxic agents. A common mechanism blocked by divalent cations.” J. Biol. Chem. 261: 9300-9308) to result in membrane depolarization due to passage of monovalent ions through the compromised pores. Specifically, efflux of K⁺, influx of Na⁺, influx of Ca²⁺ and efflux of Cl⁻ together result in depolarization of the cells as the internal and external ionic solutions rapidly equilibrate. The infusion of water results in cell swelling and the distension of pores to allow the loss of larger molecules such as intermediates of metabolism (e.g., nucleotides and sugar phosphates) altogether resulting in a catastrophic loss of cellular function and cell death, known as pyroptosis. Porin based cytolysis of red blood cells (RBC) results in the ultimate loss of large tetrameric hemoglobin or hemolysis. Cytolysis of other cell types results in the massive release of cytokines and lysosomal enzymes to result in nerve and tissue necrosis as well as profound pain and inflammation secondary to proximal cell lysis of dermal cells, including dermal mast cells, nerves and capillary blood cells.

Chironex fleckeri venom, as do all cubozoan venoms studied to date, contains extremely potent pore forming toxins. These hemolysins have not been considered to be lethal, as clinical presentations post mortem examinations have not consistently demonstrated lethal levels of hemolysis. However, it has been discovered that a catastrophic hyperkalemic state with pulseless electrical activity (PEA) precedes clinically measurable hemolysis, and furthermore, that this catastrophic hyperkalemic state is specifically caused by the cubozoan venom PFTs. In addition, stings caused by cubozoans can include significant burning pain as well as markedly obvious hemolysis at the sting site. Zinc-containing compounds, such as gluconate, have been discovered to inhibit hemolysis and the hyperkalemia that precedes hemolysis. Pore-forming toxins exhibit general classes of conserved structural homology for which some calcium appears to be involved in polymerization to form transmembrane pores. Thus, some divalent cations, such as, for example, Zn²⁺ and Mg²⁺, can competitively bind to calcium binding sites and inhibit self assembly of porin proteins to form functional polymeric pores. Zinc has also been shown to exhibit membrane-protective effects in the presence of membrane-disruptive molecules and toxins broadly classified as MPs.

In conducting research on the systematic biochemical characterization of the venoms of the cubozoan members of the cnidarian families, it has been discovered that zinc is a rapid and effective inhibitor of several specific porin-related pathogenic outcomes, including, but not limited to, hyperkalemia, hemolysis, cytokine and histamine release, and catecholamine surge. Zinc can increase survival time following lethal doses of Chironex fleckeri (the Australian box jellyfish) venom in mice. Furthermore, zinc effects can be augmented by copper to markedly reduce pain and swelling attendant to box jelly stings. Using data from these studies, the inventor has developed compositions and treatment protocols using the same that specifically inactivate individual constituents that comprise this complex venom. The findings that hemolysis-associated pain and swelling can be blocked by application of a composition containing zinc and copper is a significant advance in the emergency management of these high-morbidity stings.

Accordingly, in embodiments of the invention, methods for use of a composition that includes zinc and copper in treating topical stings caused by porin exposure and diseases, illnesses and syndromes resulting from the same are provided. In some embodiments, the zinc component is zinc gluconate. In some embodiments, the copper component is copper gluconate. In some embodiments, the composition additionally comprises at least one of the following: lactulose, magnesium sulfate, urea, or a similar compound that is a commonly recognized as safe (CRAS) therapeutic agent for topical use.

In embodiments of the invention, methods for topical administration of the compositions as disclosed herein as a specific clinical management modality for the emergency care of acute stings of cubozoans such as, for example, Chirodropidae (e.g. Chironex fleckeri, Chiropsalmus quadrumanus) and Carybdeidae (e.g. Carukia barnesi, Malo maxima, Carybdea alata, Alatina moseri) are provided.

In some embodiments of the invention, methods for transdermal administration of compositions as disclosed herein are provided.

Conditions and Diseases Associated with Exposure to Pore-Forming Toxins

In embodiments of the invention, pore-forming-toxin-related illnesses and conditions can include, but are not limited to, hemolytic and inflammatory dermal responses, acute point pain and swelling at the site of a PFT sting, hyperkalemia, hypovolemia, hypocalcemia, toxic calcium influx, hemolysis, excessive cytokine and histamine release, Irukandji syndrome, catecholamine surge, bacterial sepsis, cardiovascular collapse, pulseless electrical activity (PEA) and envenomation by cnidarians with cardiovascular collapse, respiratory distress, inflammation and/or Irukandji syndrome. For example, severe envenomations by the Carybdeidae family (four-tentacled cubozoan members of the cnidarian phylum) can lead to Irukandji syndrome, which is associated with “catecholamine surge” and “cytokine storm” type symptoms that include pain, sweating, acute anxiety, and life threatening cardiovascular effects.

Additional conditions include diseases, illnesses or syndromes associated with porin-mediated cell and tissue damage, including, but not limited to, those resulting from a bacterial infection, a viral infection, a reaction to insect stings and arachnid bites, and reactions to stings from organisms within the cnidarian phylum.

Further relevant conditions include membrane perturbation effects such as sea urchin impalement, psoriasis and dermal inflammatory responses reactions to small molecular weight compounds including alkaloids comprising certain insect venoms or certain toxic plants.

Exemplary bacteria that produce PFTs of prominent health importance include, but are not limited to, Staphylococcus, Clostridium, Streptococcus, Bacillis, Aeromonas, Escherichia, and Neisseria.

Exemplary viruses that produce PFTs of prominent health importance include, but are not limited to, viruses from the Reoviridae, Paramyxoviridae and Orthomyxoviridae families.

PFTs have been found in all members of the cnidarian phylum that have been examined including, for example, in cubozoans (or box jellyfish), hydrozoans such as Physalia sp., and stinging hydroid, in scyphozoans, stinging nettles, as well as anthozoans such as anemones, coral and fire coral

Pore-forming toxin related illnesses and conditions can be caused by the exemplary agents listed in Table 1.

TABLE 1
Agents that form divalent cation-sensitive pores (from: Bashford, C.L. Membrane
Pores - From Biology to Track-Etched Membranes. Biosci. Rep. 15 (1995) 553-565.)

| Agent | Examples |
|---|---|
| Viruses | Sendai, Newcastle Disease, Influenza |
| Bacterial toxins | S. aureus α- and δ-toxin, Streptolysin O, C. perfringens q-toxin, S. |
| | pneumoniae pneumolysin, E. coli haemolysin, A. hydropohila |
| | haemolysin, C. lacteus cytolysin, B. thuringiensis δ-endotoxin |
| Animal toxins | Melittin (honey bee), Cytolysin (anthozoans, cubozoans), Latrotoxin |
| | (spider venom) |
| Immune proteins | Activated complement, Cytolysin (perforin) |
| Synthetic compounds | Polycations, Triton X-100 |

Pore-forming toxin related illnesses and conditions can also be caused by the family of pore-forming mushroom toxins. Exemplary toxins in this family include, but are not limited to, phallolysin, flammutoxin, ostreolysin, and the cytolytic proteins identified in Berheinmer (Bernheimer, A. W., and B. Rudy. 1986. Biochim Biophys Acta 864:123-141, which is incorporated herein by reference in its entirety).

Other medically relevant porin-mediated conditions or sources of porin exposure will be apparent to a person of ordinary skill in the art.

### Zinc-Containing Compounds

In embodiments of the invention, the zinc component can be a zinc-containing compound that includes any non-toxic counter-ion to zinc. For example, the counter-ion can be any sugar-based counter-ion, including, but not limited to, acetate, malate or formulations based on oxidized sugars such as, for example, D-lactulose, glucose, lactose, galactose, sucrose, pentose, and fructose. In some embodiments, the counter-ion can be any anion, including, but not limited to, chloride, sulfate, phosphate, acetate, propionate, butyrate, oxalate, malonate, succinate, gluconate, or a complex polyanion. In some embodiments, the zinc-containing compound can be zinc gluconate.

In some embodiments, the counter-ion can be any ion selected for its property in meeting a desire to 1) avoid placing an additional ionic load in the plasma and/or 2) avoid burdening the kidney clearance load of a subject afflicted by a porin-mediated disease or condition. Applicable counter-ions that meet these criteria will be apparent to a person of ordinary skill in the art

In embodiments of the invention, the zinc component can be found in the compositions as disclosed herein in an amount ranging from about 0.1% (w/v) to about 50% (w/v). In some embodiments, the zinc component can be found in the disclosed compositions in amount ranging from about 0.5% (w/v) to about 45% (w/v). In some embodiments, the zinc component can be found in the disclosed compositions in amount ranging from about 1.0% (w/v) to about 40% (w/v). In some embodiments, the zinc component can be found in the disclosed compositions in amount ranging from about 2.0% (w/v) to about 35% (w/v). In some embodiments, the zinc component can be found in the disclosed compositions in amount ranging from about 5.0% (w/v) to about 30% (w/v). In some embodiments, the zinc component can be found in the disclosed compositions in amount ranging from about 7.5% (w/v) to about 25% (w/v). In some embodiments, the zinc component can be found in the disclosed compositions in amount ranging from about 10% (w/v) to about 20% (w/v). In some embodiments, the zinc component can be found in the disclosed compositions in amount ranging from about 12.5% (w/v) to about 17.5% (w/v). In some embodiments, the zinc component can be found in the disclosed compositions in amount of about 16% (w/v).

### Copper-Containing Compounds

In embodiments of the invention, the copper component can be a copper-containing compound that includes any non-toxic counter-ion to copper. For example, the counter-ion can be any sugar-based counter-ion, including, but not limited to, acetate, malate or formulations based on oxidized sugars, such as, for example, D-lactulose, glucose, lactose, galactose, sucrose, pentose, and fructose. In some embodiments, the counter-ion can be any anion, including, but not limited to, chloride, sulfate, phosphate, acetate, propionate, butyrate, oxalate, malonate, succinate, gluconate, or a complex polyanion. In some embodiments, the copper-containing compound can be copper gluconate.

In some embodiments, the counter-ion can be any ion selected for its property in meeting a desire to 1) avoid placing an additional ionic load in the plasma and/or 2) avoid burdening the kidney clearance load of a subject afflicted by a porin-mediated disease or condition. Applicable counter-ions that meet these criteria will be apparent to a person of ordinary skill in the art

In embodiments of the invention, the copper component can be found in the compositions as disclosed herein in an amount ranging from about 0.1% (w/v) to about 50% (w/v). In some embodiments, the copper component can be found in the disclosed compositions in amount ranging from about 0.5% (w/v) to about 40% (w/v). In some embodiments, the copper component can be found in the disclosed compositions in amount ranging from about 1.0% (w/v) to about 25% (w/v). In some embodiments, the copper component can be found in the disclosed compositions in amount ranging from about 2.0% (w/v) to about 10% (w/v). In some embodiments, the copper component can be found in the disclosed compositions in amount ranging from about 5.0% (w/v) to about 8% (w/v). In some embodiments, the copper component can be found in the disclosed compositions in amount of about 6.7% (w/v).

### Additional Compounds for Use in the Compositions

In embodiments of the invention, the composition can include a compound that enhances the effect of copper compounds and zinc compounds in reducing or alleviating conditions and diseases associated with exposure to PFTs. The compound can be one that is commonly or generally recognized as safe (GRAS) for topical use. Exemplary compounds that can be employed in the disclosed compositions include, but are not limited to, d-lactulose, magnesium sulfate, urea and the like.

In embodiments of the invention, the additional compound can be found in the compositions as disclosed herein in an amount ranging from about 0.1% (w/v) to about 95% (w/v). In some embodiments, the additional compound can be found in the disclosed compositions in amount ranging from about 0.5% (w/v) to about 85% (w/v). In some embodiments, the additional compound can be found in the disclosed compositions in amount ranging from about 1.0% (w/v) to about 75% (w/v). In some embodiments, the additional compound can be found in the disclosed compositions in amount ranging from about 2.0% (w/v) to about 65% (w/v). In some embodiments, the additional compound can be found in the disclosed compositions in amount ranging from about 5.0% (w/v) to about 55% (w/v). In some embodiments, the additional compound can be found in the disclosed compositions in amount ranging from about 10% (w/v) to about 45% (w/v). In some embodiments, the additional compound can be found in the disclosed compositions in amount ranging from about 15% (w/v) to about 35% (w/v). In some embodiments, the additional compound can be found in the disclosed compositions in amount ranging from about 20% (w/v) to about 25% (w/v).

D-lactulose can substantially inhibit hemolytic toxins and thus substantially reduce morbidity and mortality. In particular, a dramatic absence of hemolysis was observed in the presence of 10 mM D-lactulose (Chung, J. J., Ratnapala, L. A., Cooke, I. M., Yanagihara, A. A., Toxicon 39 (2001) 981-990, which is incorporated herein by reference in its entirety). In some embodiments, a composition as disclosed herein comprises D-lactulose. Thus, in some embodiments, d-lactulose can be found in the disclosed compositions in amount ranging from about 0.5% (w/v) to about 25% (w/v). In some embodiments, d-lactulose can be found in the disclosed compositions in amount ranging from about 1.0% (w/v) to about 15% (w/v). In some embodiments, d-lactulose can be found in the disclosed compositions in amount ranging from about 2.0% (w/v) to about 10% (w/v). In some embodiments, d-lactulose can be found in the disclosed compositions in amount ranging from about 4.0% (w/v) to about 5.0% (w/v).

Magnesium sulfate can inhibit self-assembly for formation of a pore. Magnesium sulfate also has the added benefit of being soluble in aqueous solutions and inexpensive as a reagent. In some embodiments, a composition as disclosed herein comprises magnesium sulfate. In some embodiments, magnesium sulfate can be found in the disclosed compositions in amount ranging from about 0.5% (w/v) to about 30% (w/v). In some embodiments, magnesium sulfate can be found in the disclosed compositions in amount ranging from about 1.0% (w/v) to about 25% (w/v). In some embodiments, magnesium sulfate can be found in the disclosed compositions in amount ranging from about 2.0% (w/v) to about 20% (w/v). In some embodiments, magnesium sulfate can be found in the disclosed compositions in amount ranging from about 5.0% (w/v) to about 15% (w/v).

Urea is a protein-denaturing agent that is safe for topical use. In some embodiments, a composition as disclosed herein comprises urea. In some embodiments, urea can be found in the disclosed compositions in amount ranging from about 5.0% (w/v) to about 90% (w/v). In some embodiments, urea can be found in the disclosed compositions in amount ranging from about 10% (w/v) to about 80% (w/v). In some embodiments, urea can be found in the disclosed compositions in amount ranging from about 15% (w/v) to about 70% (w/v). In some embodiments, urea can be found in the disclosed compositions in amount ranging from about 20% (w/v) to about 60% (w/v). In some embodiments, urea can be found in the disclosed compositions in amount ranging from about 30% (w/v) to about 50% (w/v). In some embodiments urea can be covalently modified . . . .

### Dosage

The dosage administered will vary depending upon known factors, such as the pharmacodynamic characteristics of the particular agent; the age, health and weight of the recipient; the nature and extent of the symptoms; concurrent treatment; the frequency of treatment; and the effect desired. In addition, an effective amount of a composition as disclosed herein will depend, at least, on the particular method of use, the subject being treated, the severity of the affliction, and the manner of administration of the composition. A “therapeutically effective amount” of a composition is a quantity of a composition as disclosed herein sufficient to achieve a desired effect in a subject (host) being treated. For example, this can be the amount of a composition necessary to prevent, inhibit, reduce or relieve a condition caused by a pore-forming toxin as disclosed herein.

Therapeutically effective doses of a disclosed composition can be determined by one of skill in the art. The amount of the composition that is effective in the treatment or prevention of a condition associated with a pore-forming toxin can be determined by standard clinical techniques well known to those of skill in the art. In addition, in vitro or in vivo assays can optionally be employed to help identify optimal dosage ranges. One of ordinary skill in the art will readily be able determine an approximate or precise dose to be employed.

TABLE 2
Blends

| | Compounds | low % (w/v) | high % (w/v) |
|---|---|---|---|
| Blend 1 | Zinc | 0.1 | 50.0 |
| | Copper | 0.1 | 50.0 |
| | D-lactulose | 0.0 | 25.0 |
| | Magnesium Sulfate | 0.0 | 30.0 |
| | Urea | 0.0 | 90.0 |
| Blend 2 | Zinc | 0.1 | 50.0 |
| | Copper | 0.1 | 50.0 |
| | D-lactulose | 0.5 | 25.0 |
| | Magnesium Sulfate | 0.5 | 30.0 |
| | Urea | 5.0 | 90.0 |
| Blend 3 | Zinc | 0.5 | 45.0 |
| | Copper | 0.5 | 40.0 |
| | D-lactulose | 1.0 | 15.0 |
| | Magnesium Sulfate | 1.0 | 25.0 |
| | Urea | 10.0 | 80.0 |
| Blend 4 | Zinc | 1.0 | 40.0 |
| | Copper | 1.0 | 25.0 |
| | D-lactulose | 2.0 | 10.0 |
| | Magnesium Sulfate | 2.0 | 20.0 |
| | Urea | 15.0 | 70.0 |
| Blend 5 | Zinc | 2.0 | 35.0 |
| | Copper | 2.0 | 10.0 |
| | D-lactulose | 4.0 | 5.0 |
| | Magnesium Sulfate | 5.0 | 15.0 |
| | Urea | 20.0 | 60.0 |
| Blend 6 | Zinc | 5.0 | 30.0 |
| | Copper | 5.0 | 8.0 |
| | D-lactulose | 0.0 | 0.0 |
| | Magnesium Sulfate | 0.0 | 0.0 |
| | Urea | 30.0 | 50.0 |
| Blend 7 | Zinc | 7.5 | 25.0 |
| | Copper | 6.7 | 6.7 |
| | D-lactulose | 0.0 | 25.0 |
| | Magnesium Sulfate | 0.0 | 30.0 |
| | Urea | 0.0 | 90.0 |
| Blend 8 | Zinc | 10.0 | 20.0 |
| | Copper | 0.1 | 50.0 |
| | D-lactulose | 0.0 | 25.0 |
| | Magnesium Sulfate | 0.0 | 30.0 |
| | Urea | 0.0 | 90.0 |

### Pharmaceutical Formulations

In embodiments of the invention, a therapeutic treatment is provided, the treatment comprising the use of a composition as disclosed herein, which can be a pharmaceutical composition or therapeutic agent containing the same, or a pharmaceutically acceptable salt or solvate thereof, and at least one pharmaceutical carrier or diluent. The composition or agent can be used in the prophylaxis and/or treatment of the foregoing diseases or conditions and in therapies as disclosed herein. In some embodiments, the carrier is a pharmaceutically acceptable carrier and is compatible with, i.e. does not have a deleterious effect upon, the other ingredients in the composition. The carrier can be a solid or liquid and can be formulated as a unit dose formulation, for example, as a single-use application that can contain from 0.05 to 95% by weight of the active ingredient.

In some embodiments, the composition as disclosed herein is present in the pharmaceutical composition or therapeutic agent in an amount ranging from about 0.5 percent to about 90 percent by weight of the pharmaceutical composition or therapeutic agent. In some embodiments, the composition is present in an amount ranging from about 1 percent to about 85 percent by weight of the pharmaceutical composition or therapeutic agent. In some embodiments, the composition is present in an amount ranging from about 5 percent to about 80 percent by weight of the pharmaceutical composition or therapeutic agent. In some embodiments, the composition is present in an amount ranging from about 10 percent to about 75 percent by weight of the pharmaceutical composition or therapeutic agent. In some embodiments, the composition is present in an amount ranging from about 15 percent to about 50 percent by weight of the pharmaceutical composition or therapeutic agent. In some embodiments, the composition is present in an amount ranging from about 25 percent to about 35 percent by weight of the pharmaceutical composition or therapeutic agent.

In some embodiments, the composition is present in an amount ranging from about 2 percent to about 25 percent by weight of the pharmaceutical composition or therapeutic agent. In some embodiments, the composition is present in an amount ranging from about 2 percent to about 20 percent by weight of the pharmaceutical composition or therapeutic agent. In some embodiments, the composition is present in an amount ranging from about 2 percent to about 10 percent by weight of the pharmaceutical composition or therapeutic agent. In some embodiments, the composition is present in an amount ranging from about 5 percent to about 15 percent by weight of the pharmaceutical composition or therapeutic agent. In some embodiments, the composition is present in an amount ranging from about 5 percent to about 10 percent by weight of the pharmaceutical composition or therapeutic agent.

In some embodiments, the pharmaceutical composition is a solution.

In some embodiments, the pharmaceutical composition can be administered topically, such as, for example, by a solution, a spray, a lotion, an ointment, a cream, or a patch.

In some embodiments, the pharmaceutical composition can be administered transdermally, such as, for example, by a transdermal patch.

In some embodiments, a therapeutic composition formulation as disclosed herein can contain at least one additional agent, including, but not limited to, a carrier, an adjuvant, an emulsifying agent, a suspending agent, a flavoring, a perfume, a binding agent, or the like.

As used herein, “pharmaceutically acceptable carrier” and “carrier” generally refer to a non-toxic, inert solid or non-inert, semi-solid or liquid filler, diluent, encapsulating material or formulation auxiliary of any type. Some non-limiting examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil; kukui nut oil, camphor oil; and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; and phosphate buffer solutions, as well as other non-toxic compatible lubricants such as sodium lauryl sulfate and magnesium stearate, as well as coloring agents, releasing agents, coating agents, sweetening, flavoring, menthol and perfuming agents, preservatives and antioxidants can also be present in the composition, according to the judgment of the formulator.

The pharmaceutically acceptable carriers described herein, for example, vehicles, adjuvants, excipients, or diluents, are well-known to those who are skilled in the art. Typically, the pharmaceutically acceptable carrier is chemically inert to the therapeutic agents and has no detrimental side effects or toxicity under the conditions of use. The pharmaceutically acceptable carriers can include polymers and polymer matrices, nanoparticles, microbubbles, and the like.

The therapeutic treatment can further comprise inert diluents such as water or other solvents, solubilizing agents and emulsifiers such as ethyl alcohol, isopropyl alcohol, ethyl carbonate, ethyl acetate, benzyl benzoate, propylene glycol, 1,3-butylene glycol, dimethylformamide, oils (in particular, cottonseed, groundnut, corn, germ, olive, castor, and sesame oils), glycerol, polyethylene glycols and fatty acid esters of sorbitan, and mixtures thereof.

Polymerizing agents, such as, for example, polyvinyl alcohols are also contemplated for inclusion in compositions and formulations as disclosed herein.

### Routes and Forms of Administration

In embodiments of the invention, a composition as disclosed herein can be administered topically or transdermally. Such routes of administration can be optimized according to the applicable clinical scenario.

In embodiments of the invention, the therapeutic composition is administered topically. In some embodiments, topical administration of the therapeutic composition is accompanied by application of heat to increase dermal adsorption. Heat can be applied by, for example, immersion of the affected area in hot water subsequent to topical administration of the composition. In some embodiments, heat is applied by immersion in heated water of up to 45° C. for at least 5 minutes. In some embodiments, heat is applied by immersion in heated water of up to 45° C. for at least 10 minutes. In some embodiments, heat is applied by immersion in heated water of up to 45° C. for at least 15 minutes. In some embodiments, heat is applied by immersion in heated water of up to 45° C. for at least 20 minutes. In some embodiments, heat is applied by immersion in heated water of up to 45° C. for at least 30 minutes. In some embodiments, heat is applied by immersion in heated water of up to 45° C. for at least 45 minutes. In some embodiments, heat is applied by immersion in heated water of up to 45° C. for at least 60 minutes.

In embodiments of the invention, the therapeutic composition is administered transdermally. Typically, the composition is applied to the drug electrode of an iontophoresis unit, and the drug electrode and ground electrode are applied to the skin of a subject in need of treatment. Voltage is then applied to deliver the compound transdermally to the subject. Typical composition concentrations applied to the drug electrode range from about 0.1 mM to about 250 mM, from about 0.5 mM to about 200 mM, from about 1 mM to about 100 mM, from about 2.5 mM to about 50 mM, or from about 5 mM to about 25 mM. Typical voltages applied to the skin of the subject range from about 0.1 mAmp/min to about 80 mAmp/min. An appropriate voltage amount is one that alleviates symptoms associated with exposure to a pore-forming toxin and the improves medical outcome for the subject while maintaining the comfort level of the subject being treated.

Formulations suitable for transdermal administration can be prepared for delivery by transdermal patches with or without electrophoretic current to augment diffusion or deliver agent. Transdermal administration can be also by use of “nanoneedles”. (see Escobar-Chávez J J, Bonilla-Martínez D, Villegas-González M A, Revilla-Vázquez A L. J. Clin. Pharm (2009), which is incorporated by reference in its entirety).

The methods of treatment of the present invention include methods that are administered to a subject in need thereof. As used herein, “subject,” may refer to any living creature, typically an animal, preferably a mammal, and more preferably a human.

In some embodiments, the therapeutic composition is used as a prophylactic treatment prior to a subject coming in contact with an agent that causes the reactions, symptoms or conditions disclosed herein. In some embodiments, the therapeutic composition is used as a topical or oral prophylactic treatment prior to a subject encountering a cnidarian.

In some embodiments, oral zinc gluconate is used as a prophylactic to build up serum levels and provide a protective level of zinc prior to a potential exposure to PFTs or MPs.

Suitable pharmaceutical carriers are described in Remington's Pharmaceutical Sciences, Mack Publishing Company, a standard reference text in this field.

### Combination Therapy

In embodiments of the invention, the methods disclosed herein further comprise a combination therapy, wherein at least one additional therapeutic agent is administered to the patient. In some embodiments, the at least one additional therapeutic agent is selected from the group consisting of antibiotics (such as penicillin, tetracycline, and Tobramycin) to control infection, D-lactulose, specific phospholipase inhibitors useful in certain types of envenomations, steroids, and pain relievers/anti-inflammatory agents (such as ibuprofen).

The therapeutic methods of the invention can be administered by any conventional method available for use in conjunction with pharmaceutical drugs, either as individual therapeutic agents or in a combination of therapeutic agents.

It will be appreciated that the methods of the combining a therapeutic composition as disclosed herein with an additional treatment can be administered: (1) simultaneously by combination of the compounds in a co-formulation or (2) by alternation, i.e. delivering the compounds serially, sequentially, in parallel or simultaneously in separate pharmaceutical formulations. In alternation therapy, the timing of administration of the second, and optionally a third active ingredient, is such that there is no loss of benefit of any synergistic therapeutic effect of the combination of the active ingredients. In some embodiments, by either method of administration (1) or (2), the combination is preferably administered to achieve the most efficacious results. In some embodiments, by either method of administration (1) or (2), the combination is administered to achieve peak plasma concentrations of each of the active ingredients.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### A Zinc-Containing Compound Inhibits the Effects of Cnidarian Venom in Whole Blood and Isolated Red Blood Cells

Concentration gradient driven monovalent ion flux, or more specifically, potassium (10 efflux into plasma accompanied by both sodium (Na⁺) influx and chloride ion (Cl⁻) efflux, as well as divalent cation toxic influx (Ca²⁺), can occur in cubozoan envenomation with sufficiently rapid kinetics, and this can lead to cardiotoxic calcium influx with lethal plasma hyperkalemia at rates beyond the kidney clearance rate in an affected subject. Ex vivo assays of whole human blood demonstrated that profound hyperkalemia results from Chrionex PFT exposure with lethal levels of free plasma potassium (>10 mM). In the following examples, a therapeutic composition containing the components as listed below in Table 3 was used or administered in in vitro experiments or in subject testing trials.

TABLE 3

| Therapeutic | | | % (wt of |
|---|---|---|---|
| composition for | | | component/ |
| treatment of jellyfish | Stock | Composition | volume of |
| stingsComponent | Concentration | Concentration | composition) |
| Zinc gluconate | 350 mM | 16 g/100 mL | 16% |
| Copper gluconate | 150 mM | 6.7 g/100 mL | 6.7% |
| D-lactulose | 125 mM | 4.3 g/100 mL | 4.3% |
| Magnesium sulfate | 550 mM | 13 g/100 mL | 13% |
| Urea | 7 M | 44 g/100 mL | 44% |

### Example 2

### Combinatorial Effect of Zinc and Copper in Inhibiting Venom-Induced Hemolysis

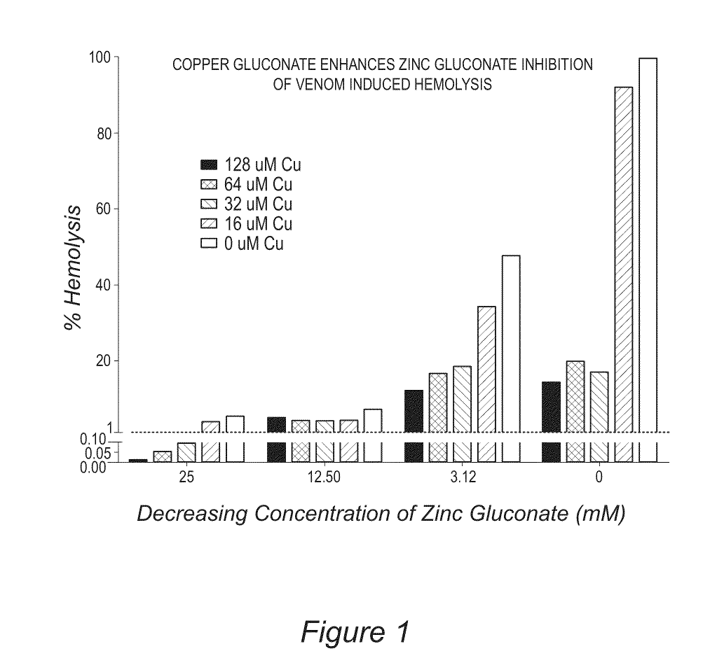

Using a 96-well plate, human washed red blood cells (RBC, 2%) were subjected to Alatina moseri venom (6.4 U/mL/%). And treated simultaneously with a dose of zinc gluconate administered to a final concentration of from 0 to 25 mM zinc gluconate in combination with a dose of copper gluconate administered to a final concentration of from 0 to 128 μM. Cells were incubated with the amounts of zinc gluconate and/or copper gluconate as indicated in FIG. 1 for 1 hour at 37° C. At the end of the incubation period, the 96 well plate was centrifuged briefly at 4° C. to pellet red blood cells from plasma. Plasma hemoglobin levels were then determined spectrophotometrically.

As shown in FIG. 1, the results indicate that, between 0 mM and 12.5 mM of zinc gluconate, the presence of copper gluconate substantially increased the baseline inhibition of venom-induced hemolysis by zinc gluconate. At concentrations of 25 mM zinc gluconate, addition of up to 128 μM copper gluconate practically eliminated hemolysis after exposure to venom. The results illustrate that the presence of copper can enhance the effects of zinc in inhibiting venom-induced hemolysis to the point of almost complete inhibition of hemolysis.

### Example 3

### A Composition Containing Copper and Zinc Effectively Inhibits Venom-Induced Hemolysis In Vitro

In this example, human red blood cell (RBC) agar was exposed to Alatina moseri venom in the presence of 100 μL of an exemplary composition as disclosed in Table 3 or 100 μL of a commercially available product (Jelly Squish®). Human RBC agar (2% RBC, 3% NuSieve GTG low melting agarose and 150 mM NaCl) were exposed to 2 mm sections of fresh, live Alatina moseri tentacle sections. Two (2) to five (5) minutes after exposure to tentacle tissue, 100 μL of the exemplary composition or commercial product were administered to the RBC samples. The samples were then incubated at 37° C., and photographs were taken at 15 minute intervals starting at t=0.

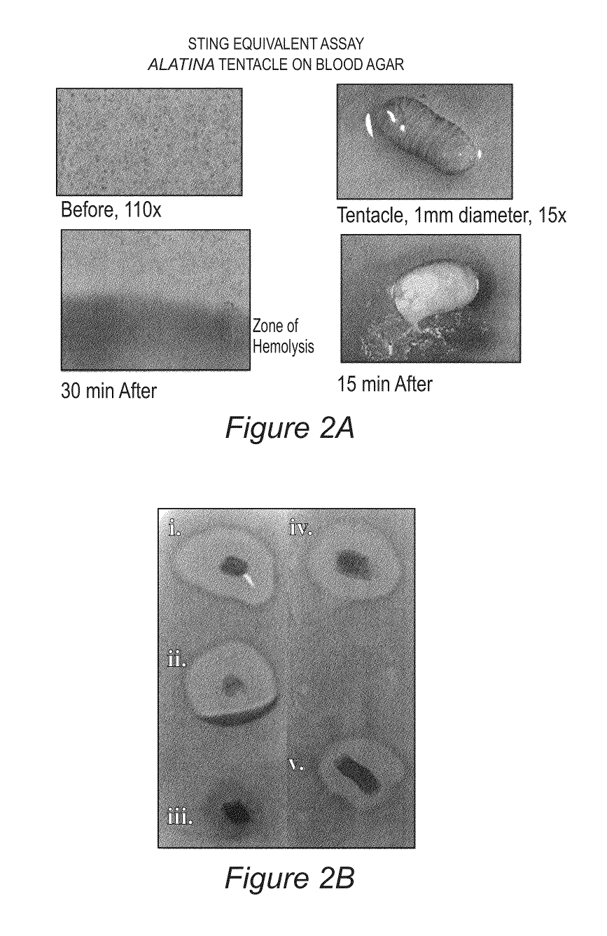

FIG. 2A illustrates the effect of tentacle tissue on RBC agar without any administration of anti-venom composition. Clear zones of hemolysis in the RBC agar are evident in the area immediately surrounding the tentacle tissue within 30 minutes of tentacle exposure.

FIG. 2B illustrates the effect of treatment with the exemplary composition and the commercial product upon the tentacle-exposed RBC agar. As shown in sections (i), (ii) and (iii) of the photograph, the application of the exemplary composition was effective in preventing formation of the clear zones of hemolysis surrounding the tentacle tissue, whereas application of the commercial product (sections (iv) and (v)) did not prevent the zone of hemolysis from forming.

Thus, this experiment illustrates the effectiveness of the exemplary composition over a commercially available product in inhibiting venom-induced hemolysis.

### Example 4

### A Composition Containing Copper and Zinc Effectively Alleviates Point Pain in a Subject Exposed to a Venom Sting

A human volunteer was exposed to Alatina moseri live tentacle tissue and treated ten (10) minutes after exposure with topical administration of an exemplary composition as described in Table 3 or with a commercially available product (Jelly Squish®). Point pain and visual signs of hemolysis were monitored in the exposed and treated skin areas of the subject for 24 hours.

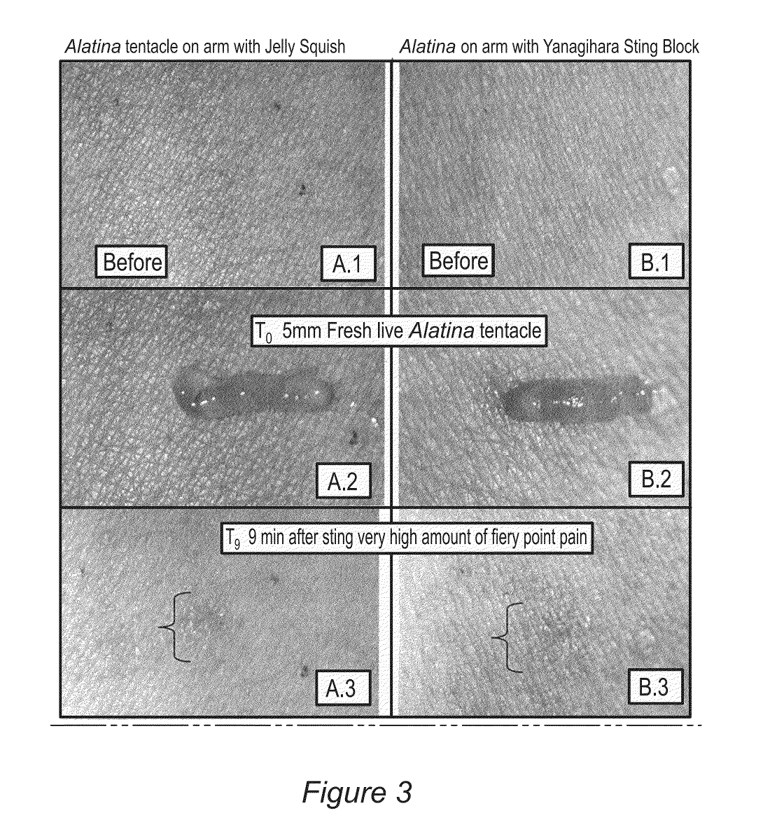

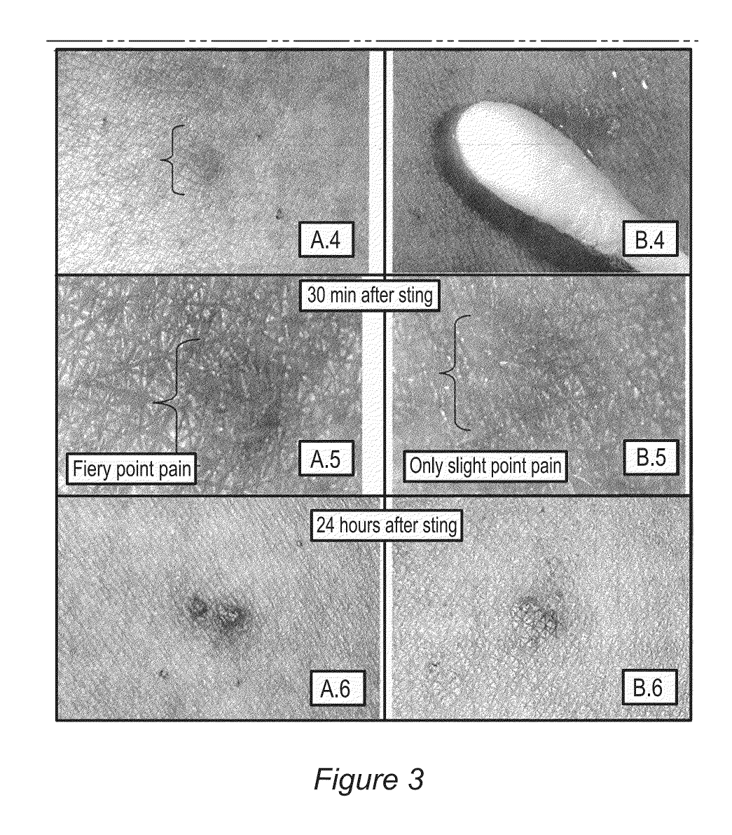

As shown in FIG. 3, the subject experienced very high amounts of what was described as “fiery” point pain within minutes of exposure to the tentacle tissue. After topical treatment with the Jelly Squish® commercial product, although there was transient momentary, relief, the subject continued to experience burning point pain, and visual signs of hemolysis were apparent at 30 minutes and 24 hours after venom exposure (i.e. the venom-exposed skin area treated with the product continued to look very red). In comparison, after topical treatment with the exemplary composition, the subject experienced an alleviation of pain symptoms and described the sensation as “slight” point pain 10 minutes after application. In addition, the venom-exposed skin area treated with the exemplary therapeutic composition appeared only slightly red at 30 minutes and 24 hours after venom exposure, indicating a less extreme degree of hemolysis compared to the venom-exposed skin area treated with commercial product.

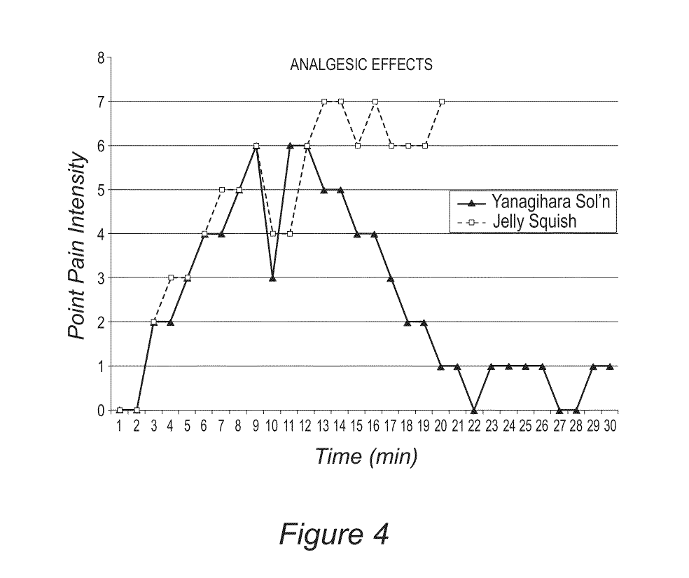

FIG. 4 illustrates the intensity of point pain experienced by the subject during immediately after exposure to tentacle tissue and prior to administration of the topical compositions as well as subsequent to administration of the compositions. Before the compositions were applied, the subject experienced an increase in the intensity of point pain at the site of venom exposure. Subsequent to application of the exemplary therapeutic composition, the subject experienced a gradual drop in point pain intensity over a period of 10 minutes, whereas application of the commercial product produced no relief from point pain intensity.

Thus, the results of this experiment show that the exemplary therapeutic composition is dramatically more effective than a commercially available product in alleviating pain associated with porin exposure.

### Example 5

### Use of a Composition Containing Copper and Zinc to Treat a Human Subject Exposed to a Cubozoan Pore-Forming Toxin (Topical Administration)

In this Example, human subjects are treated with an exemplary composition containing zinc and copper as disclosed herein after being envenomated by a cubozoan porin. Human subjects are topically administered a composition containing zinc gluconate and copper gluconate immediately after porin exposure, with additional application of the composition administered according to the subjects' comfort level. Administration of the composition is observed to reduce the severity of point pain and physiological symptoms (including hemolysis) associated with the venom poisoning and to decrease the likelihood of scarring occurring on the venom-exposed skin.

### Example 6

### Use of a Composition Containing Zinc and Copper to Treat a Human Subject Exposed to a Cubozoan Pore-Forming Toxin (Transdermal Administration)

In this Example, human subjects are treated with an exemplary composition containing zinc and copper as disclosed herein via a transdermal patch after being envenomated by a cubozoan porin. Human subjects afflicted by porin exposure are quickly administered a composition containing zinc gluconate and copper gluconate by a transdermal patch using an iontophoresis unit set to deliver a 5 mM solution of zinc gluconate at 40 mAmp/min. Administration of the composition is observed to reduce the severity of point pain and physiological symptoms, including hemolysis, associated with the venom poisoning and to decrease the likelihood a scarring in the venom-exposed skin.

### Example 7

### Use of a Composition Containing Zinc and Copper to Treat a Human Subject Exposed to a Bacterial Pore-Forming Toxin (Topical Administration)

In this Example, human subjects are treated with an exemplary composition containing zinc and copper as disclosed herein after being exposed to a bacterial porin. Human subjects are topically administered a composition containing zinc gluconate and copper gluconate immediately after porin exposure, with additional application of the composition administered according to the subjects' comfort level. Administration of the composition is observed to reduce the severity of physiological symptoms associated with porin exposure and to improve the health of the subject.

### Example 8

### Use of a Composition Containing Zinc and Copper to Treat a Human Subject Exposed to a Viral Pore-Forming Toxin (Topical Administration)

In this Example, human subjects are treated with an exemplary composition containing zinc and copper as disclosed herein after being exposed to a viral porin. Human subjects are topically administered a composition containing zinc gluconate and copper gluconate immediately after porin exposure, with additional application of the composition administered according to the subjects' comfort level. Administration of the composition is observed to reduce the severity of physiological symptoms associated with porin exposure and to improve the health of the subject.

### Example 9

### Use of a Composition Containing Zinc and Copper to Treat a Human Subject Exposed to a Mushroom Pore-Forming Toxin (Topical Administration)

In this Example, human subjects are treated with an exemplary composition containing zinc and copper as disclosed herein after being exposed to a mushroom pore-forming toxin. Human subjects are topically administered a composition containing zinc gluconate and copper gluconate immediately after porin exposure, with additional application of the composition administered according to the subjects' comfort level. Administration of the composition is observed to reduce the severity of physiological symptoms associated with porin exposure and to improve the health of the subject.

### Example 10

### Combinatorial Effect of Zinc and Copper in Inhibiting Fire Ant Venom-Induced Hemolysis

In this Example, human red blood cells (RBCs) are treated with ant venom proteins (A) and piperidine class compounds (B) over a realistic sting equivalent dose range at 37° C. for 1-8 hrs to determine the dose at which 50% of RBCs are lysed (HD₅₀ values). Based upon the determined dose range, blocking agents including but not limited to those that comprise the cubozoan sting blocking solution, are added before, with or after the addition of A and B ant constituents to the human RBCs over a sting equivalent range to test for effect. Combinatorial experiments are conducted to confirm synergistic effects of blocking agents to augment inhibition of hemolysis as a measure of cell membrane integrity.

### Example 11

### Combinatorial Effect of Zinc and Copper in Inhibiting Urchin Impalement-Induced Inflammation

In this Example, human red blood cells (RBCs) are treated with ground urchin spine material (A) and organic extract of spine material (B) over a realistic exposure dose range at 37° C. for 1-8 hrs to determine the dose at which 50% of RBCs are lysed (HD₅₀ values). Based upon the determined dose range, blocking agents including those that comprise the cubozoan sting blocking solution, are added before, with or after the addition of A and B constituents to the human RBCs over a sting equivalent range to test for effect. Combinatorial experiments are conducted to confirm synergistic effects of blocking agents to augment inhibition of hemolysis as a measure of cell membrane integrity. Cytokine release assays are also performed. Cytokine assays are a classical method of quantifying the amount of inflammation occurring as a result of even minor membrane disruption. Freshly drawn human whole blood is used to purify “buffy coat” peripheral blood monocytes (PBMC) to test the efficacy of zinc and or copper to inhibit A and B induced cytokine release. Dose testing is performed wherein a range of A and B is incubated with whole peripheral blood monocytes (PBMC) and time course release of cytokines is measured using a Luminex plate based assay in the presence or absence of zinc and copper gluconate over a feasible dose range. Phospholipase activity assay is also performed using NBD labeled phosphatidyl choline in the presence of various concentrations of B with or without a dose range of blocker constituents (namely zinc gluoconate, copper gluconate, urea, D-lactulose and Mg SO4).

### Example 12

### Use of a Composition Containing Zinc and Copper to Treat a Human Subject Exposed to Urchin Impalement-Induced Inflammation

A human subject was impaled by a sea urchin. Approximately 45 minutes after contact, most of the affected area was immersed in a blocking agent composition comprising zinc gluconate and copper gluconate. Within 5 to 10 minutes, there was a marked reduction in pain in the treated area of the wound, which was estimated by the human subject as a 90% reduction in pain. The untreated area continued to throb with pain, and was inflamed and painful for about two weeks after impalement. The treated portion of the injury healed faster despite the wounds being more severe in that area.

### Example 13

### Use of a Composition Containing Zinc and Copper to Treat a Human Subject Exposed to Spider Venom

In this Example, human subjects are treated with an exemplary composition containing zinc and copper as disclosed herein after being exposed to spider venom. Human subjects are topically administered a blocking agent composition comprising zinc gluconate and copper gluconate immediately after spider bite exposure, with additional application of the composition administered according to the subjects' comfort level. Some human subjects completely immerse the afflicted area in the blocking agent. Administration of the composition is observed to reduce the severity of physiological symptoms associated with porin exposure and to improve the health of the subject. Furthermore, administration of the composition is observed to speed healing and recovery time relative to untreated areas, even despite the higher severity of the wound in the treated areas.

### Example 14

### Use of a Composition Containing Zinc and Copper to Treat a Human Subject Exposed to Poison Ivy

In this Example, human subjects are treated with an exemplary composition containing zinc and copper as disclosed herein after being exposed to poison ivy. Human subjects are topically administered a blocking agent composition comprising zinc gluconate and copper gluconate immediately after exposure, with additional application of the composition administered according to the subjects' comfort level. Some human subjects completely immerse the afflicted area in the blocking agent. Administration of the composition is observed to reduce the severity of physiological symptoms associated with porin exposure and to improve the health of the subject. Furthermore, administration of the composition is observed to speed healing and recovery time relative to untreated areas, even despite the higher severity of the wound in the treated areas.

What is claimed:

1. A composition comprising zinc and copper in a form and an amount effective for treatment of membrane perturbation.

2. The composition of claim 1, wherein the zinc comprises a zinc-containing compound that includes a non-toxic counter-ion to zinc.

3. The composition of claim 1, wherein the copper comprises a copper-containing compound that includes a non-toxic counter-ion to copper.

4. The composition of claim 2 or 3 wherein the counter-ion comprises an ion selected from the group consisting of: acetate, malate, D-lactulose, glucose, lactose, galactose, sucrose, pentose, fructose, chloride, sulfate, phosphate, acetate, propionate, butyrate, oxalate, malonate, succinate, gluconate, and a complex polyanion.

5. The composition of claim 1 further comprising at least one of lactulose, magnesium, and urea.

6. The composition of claim 5, wherein the lactulose comprises d-lactulose.

7. The composition of claim 5, wherein the magnesium comprises magnesium sulfate.

8. The composition of claim 1, wherein the membrane perturbation comprises exposure to a pore-forming toxin.

9. The composition of claim 1, wherein the membrane perturbation comprises at least one of: cnidarian envenomation, sea urchin impalement, psoriasis, and a dermal inflammatory reaction to an alkaloid of an insect venom or a toxic plant.

10. A method of treating exposure to a pore-forming toxin (PFT) in a subject, comprising:
administering a therapeutically effective amount of the composition of claim 1,
wherein administration of a therapeutically effective amount of the composition results in treatment of a symptom or a condition resulting from PFT exposure.

11. Use of a therapeutically effective amount of the composition of claim 1 in the manufacture of a medicament for the treatment of PFT exposure.
